(19) United States
(12) Patent Application Publication
(10) Pub. No.: US 2013/0310728 A1
(43) Pub. Date: Nov. 21, 2013

(54) Device for dissecting an eye for the introduction of photosensitizer and method of refractive surgery

(71) Applicants: Theo Seiler, Zuerich (CH); Theo G. Seiler, JR., Zuerich (CH); Johannes Krause, NUMBERG (DE)

(72) Inventors: Theo Seiler, Zuerich (CH); Theo G. Seiler, JR., Zuerich (CH); Johannes Krause, NUMBERG (DE)

(21) Appl. No.: 13/473,004

(22) Filed: May 16, 2012

(51) Int. Cl. A61F 9/008; A61M 37/00

(52) U.S. Cl. 604/20

(57) ABSTRACT

A device for dissecting an eye for the introduction of photosensitizer into the cornea where laser radiation is focused in the interior of the cornea to create cavitation bubbles, whereby channels are created in the cornea through which the photosensitizer can be introduced into the cornea. Furthermore, a method for refractive surgery includes utilizing a sensitizer for hardening corneal tissue by cross-linking.

### BACKGROUND OF THE INVENTION

1. Field of the Invention

The present invention relates to refractive surgery, in particular LASIK, and related ophthalmological procedures.

2. Description of the Prior Art

In ophthalmology the technique of using a photosensitizer and electromagnetic radiation to change the biomechanical and biochemical properties of eye tissue, in particular the cornea, for therapeutic purposes has been known for more than 10 years.

The human eyeball is bounded by the corneosclera. Due to the internal eye pressure the corneosclera, which contains collagen, is under tension and confers an approximately spherical shape to the healthy eyeball. In the posterior eyeball region, the corneosclera consists of the white sclera. The cornea, which is transparent to visible light, is situated in the anterior region.

Deformations of the corneosclera can cause ametropia. For example, one type of myopia, axial myopia, can be the consequence of a sclera longitudinal expansion of the eyeball. An ellipsoidal shaped corneal surface can result in a form of astigmatism which is also called irregular corneal curvature. Another defect of the cornea is referred to as keratoconus. Here, a pathological softening of the cornea leads to a progressive thinning and cone-shaped deformation of the cornea. As the bulging increases, the cornea becomes thinner below the center. It can fracture and become scarred. This permanently reduces the visual acuity. The causes of keratoconus are still largely unknown today. It affects some families more than others, which, with other evidence, indicates a genetic disposition. Atopies, such as allergic disorders, constitute a further risk factor for the formation of a keratoconus.

The conventional therapy for an advanced keratoconus is to remove the defective cornea and replace it with an allogenic transplant. Such an operation is, however, an organ transplant, with the risks and complications this involves. Adequate vision is often not achieved until about 2 years after the operation. Furthermore, the recipients of a corneal transplant in the case of keratoconus are mostly young people, which means that the transplant must function perfectly for decades.

One therapy for keratoconus stabilizes the cornea by cross-linking. Appropriate devices for treating the corneosclera are known from the references WO 2007/128581 A2 and WO 2008/000478 A1.

EP 1 561 440 B1 describes a device wherein, with a relatively complex setup, a homogeneous distribution of the radiation is generated in the ocular tissue. There a shaped body is placed on the cornea so as to give it a desired shape while the solidity of the ocular tissue is changed using electromagnetic radiation and the photosensitizer. Such a shaped body can also be employed in connection with certain embodiments of the invention.

A device according to WO 2007/128581 A2 serves to consolidate the sclera at the posterior part of the eye. The primary radiation can here act on the sclera through the interior of the eye or through cushions applied externally. A cross-linking in the sclera is achieved by means of a photomediator or photosensitizer. In this way growth of the sclera and the progress of the axial myopia is prevented.

EP 1 854 438 A1 describes an ophthalmologic device for preventing myopia that strengthens the sclera using a photosensitizer.

The publication WO 2008/000478 A1 describes a radiation system for the biomechanical stabilization of the cornea. Here, too, a cross-linking at the cornea can be achieved in combination with a photosensitizer. The radiation system offers the possibility of treating specific ailments such as keratoconus.

US Pat. Publ. No. 2009/187171 A1 describes the creation of incisions in volumes within the stroma, where the volumes are completely contained within the stroma. The purpose is to change the shape of the cornea as a result of the intraocular pressure.

### SUMMARY OF THE INVENTION

Certain embodiments relate to a device for dissecting an eye for the introduction of a photosensitizer into eye tissue, including a source for laser radiation, a system for guiding and focusing the laser radiation relative to the eye tissue, and a computer for controlling the aforementioned system. Certain embodiments also relate to an appropriate method for dissecting an eye for the introduction of photosensitizer using laser radiation. The form and/or the mechanical properties of eye tissue, particularly of the cornea and generally of the sclera, may be changed by introducing a photosensitizer and applying electromagnetic radiation.

There are complex dependencies that discourage routine use of cross-linking therapy on the eye tissue. The relationship between the dosage used and its effect in the eye tissue are wide-ranging. In certain embodiments, dosage may refer to the intensity of the electromagnetic radiation and its distribution in space and time, and the chemical structure, concentration and action in space and time of the photosensitizer. The effects of different dosages on and in the eye tissue of a patient are strongly dependent on the patient's characteristics (measurement data). In particular, the effect of an improper dosage of the cross-linking produced by the radiation and the photosensitizer can also be undesirable, and may even result in the eye tissue being damaged or the functioning of the eye being impaired.

In certain embodiments, riboflavin may be the photosensitizer in many respects. To introduce riboflavin into the cornea it is necessary in the known techniques for the corneal epithelium to be removed at least partially since it hinders the riboflavin from penetrating the cornea, i.e., acts so to speak as a barrier to the diffusion of the riboflavin molecules into the cornea. The removal of the epithelium is, however, usually painful for the patient and the subsequent healing process is not always free from complications.

It is the object of certain embodiments to provide a device of the type cited at the beginning which enables the photosensitizer to be carefully introduced into the eye tissue, in particular with respect to the depth. A special requirement is also that the cross-linking in the eye tissue should be easy to control.

For this purpose certain embodiments provide a device according to claim 1 for dissecting an eye for the introduction of the photosensitizer into eye tissue.

As a result it is possible to introduce the photosensitizer into one or more channels quite simply without having to remove or open up significant parts of the epithelium.

The laser radiation used to create the channel or channels can be created by any suitable systems. The sources of laser radiation needed to create the channels are known e.g. from the so-called femto-LASIK. There the laser radiation of e.g., a femtosecond, picosecond, nanosecond, or attosecond laser is used as a so-called “laser scalpel” in order to cut eye tissue through “vaporization” (cavitation bubbles) with the energy of the laser light. In femto-LASIK the so-called flap cut is created with the laser scalpel, i.e. a small piece is cut out of the epithelium from the side and flipped aside so as to then perform an ablation in the exposed stroma to reshape the cornea using e.g., an excimer laser. Pulsed lasers with pulse lengths in the picosecond range and in the nanosecond range, femtosecond range, or attosecond range are also suitable for creating the channels.

The term “channel” in the sense of certain embodiments does not mean an incision area for creating a so-called flap, as this is known in femtosecond-LASIK.

The system for guiding and focusing the laser radiation relative to the eye tissue which is used in certain embodiments can be adopted from this technique. According certain embodiments the computer controlling the optical system for guiding and focusing the laser radiation, however, is so programmed that the foci of the laser radiation are moved after one another along a straight or curved line in such a way that so-called cavitation bubbles in the tissue produce a channel or a plurality of channels, which starting at the surface of the eye tissue, particularly the cornea, reach into the interior of the eye tissue, so that a photosensitizer which is brought to the entrance of the channel can enter the channel and thus penetrate into the interior of the eye tissue. The channels are here created in such a way that the separation of the individual adjacent cavitation bubbles from each other (“spacing”) is such that the structure and stability of the tissue are as little impaired as possible. On the other hand, however, the separation between the cavitation bubbles forming the channel should be so small that the photosensitizer, e.g. riboflavin, introduced into the channel in the form of a solution penetrates into the tissue through the channel in the desired manner, i.e. so to speak from cavitation bubble to cavitation bubble. In the regions between adjacent cavitation bubbles the photosensitizer solution therefore penetrates by diffusion. In certain examples, the distance between adjacent cavitation bubbles may in the range from 1 to 50 pm, for example, in the range from 5 to 30 pm, for example, in the range from 10 to 20 pm. It follows that in the sense of certain embodiments the term “channel” is not necessarily to be thought of as a continuous cavity fully free of tissue, although on the other hand completely continuous channels can also be envisaged in the sense of certain embodiments.

According to a variant of certain embodiments the channel can also follow a curved line, since by focusing the laser radiation in the interior of the tissue with focus points along the curved line a channel shape which departs from the straight line can also be created. For all the named channel shapes the channel can be created with the desired diameter and the desired geometrical configuration through the sequencing of the foci of the laser radiation with sufficiently dose separation through the so called “photodisruption”.

Certain embodiments also make it possible to adjust different densities of the channels in the eye tissue depending on the location in the eye tissue, i.e. to place more channels at preferred locations in the eye tissue than at others, a consequence of which is that where the channel density is greater there is a higher density of the photosensitizer in the tissue and therefore the biomechanical and biochemical effect at such locations is different to that at those locations in the eye tissue where the channel density is less.

Additionally, the density of the photosensitizer in the eye tissue which is finally effective can be controlled by varying the depth of the channels in the cornea depending on position.

Equivalently, the density of the photosensitizer to be introduced into the eye tissue can be controlled by choosing a larger or smaller cross-section for the one or the plurality of channels.

The width of a channel may lie in the range from 0.1 mm to 1.2 mm, although every subinterval therein is also disclosed here.

When the word “channel” appears here, the singular or the plural are to be understood.

With the device according to certain embodiments a channel system can therefore be created in the cornea that enables the interior of the cornea to be accessed from the outside. The photosensitizer solution can then be injected so that it distributes itself in the corneal stroma. For this purpose the channel system can have one or more channels, depending on the ophthalmological indication, whereby e.g., if a homogeneous distribution of the photosensitizer is required, the density of the channels (i.e. the number of channels per unit area or per unit volume) is substantially homogeneous in the region of the cornea being treated. For example, four channels can be placed in the four corneal segments, corresponding to the four segments of the projection of the cornea onto a plane. The channel system can also be generated stochastically.

Furthermore, by appropriately controlling the foci of the laser radiation, the cross-sections of the individual channels can be shaped as desired, e.g. as circles, rectangles, squares, or also as ovals or slits.

In certain embodiments the orientation of the channels is mainly transverse to the axis (A) of the eye. The term “transverse” here signifies substantially “radial.” “Radial” means directed outwards starting from the apex of the cornea.

One embodiment is so designed that the channel or channels essentially traverse the whole radial area of the cornea with substantially uniform channel density. This means, in other words, that in at least one specified area at a specified depth of the cornea, photosensitizer is brought into the corneal tissue homogeneously (uniformly with the same density) by diffusion.

Provision is made for the single channel or the plurality of channels being connected to more than one opening, this opening reaching into the surface of the eye tissue, so that photosensitizer can be brought without hindrance into the channel or channels, e.g. with a fine syringe or something similar.

These openings through which the channels are accessible from outside may be arranged at or near the border of the cornea, i.e. at or near the limbus.

The creation of channels or cavities in the sense of certain embodiments differs therefore from the creation of a so-called flap for LASIK, i.e. the channels or cavities according to the present invention do not lead to the creation of a flap which can be flipped to the side.

In another embodiment of certain embodiments, at least some of the channels are spiral-shaped.

In all the developments and embodiments of a gas, especially air, can also be injected into one or more channels or cavities.

According to a further variant, a method is taught combining the afore-mentioned method of introducing a photosensitizer into a cornea of an eye with refractive surgery performed at the cornea, in particular refractive surgery in the form of LASIK. It has been found that LASIK can be improved essentially if, before LASIK is performed, a sensitizer as described above is introduced into the cornea. By cross-linking the mechanical properties of the cornea are changed such that better results are obtained when performing, in particular, LASIK.

The afore-described hardening by cross-linking of corneal tissue can be performed by means of said sensitizer which causes said cross-linking. The sensitizer can be a photosensitizer activated by electromagnetic radiation. The electromagnetic radiation is radiated into the corneal tissue for initiating the cross-linking. Thereafter, the hardened cornea is subjected to refractive surgery by laser, in particular LASIK. Also sensitizer can be used not requiring specific activation by irradiation with a specific source of electromagnetic radiation, like a laser. Rather, such sensitizers cause cross linking without the need of a specific device delivering electromagnetic radiation for irradiating the cornea.

Further developments are described in the additional dependent claims.

### BRIEF DESCRIPTION OF THE DRAWINGS

FIG. 1 shows schematically a device for dissecting an eye for the introduction of a photosensitizer into eye tissue.

FIG. 2 shows a plan view of a cornea with a schematic description of the creation of channels therein.

FIG. 3 shows another embodiment where the computer is so programmed that it creates approximately sector-of-a-circle-shaped channel structures in the eye tissue, chiefly for treating astigmatism.

FIG. 4 shows an axial sectional view of a cornea with channels whose paths are at different depths relative to the surface of the cornea.

FIG. 5 shows an axial plan view of a cornea wherein at least one ring is formed as a channel for treating hyperopia.

### DETAILED DESCRIPTION OF THE PREFERRED EMBODIMENT

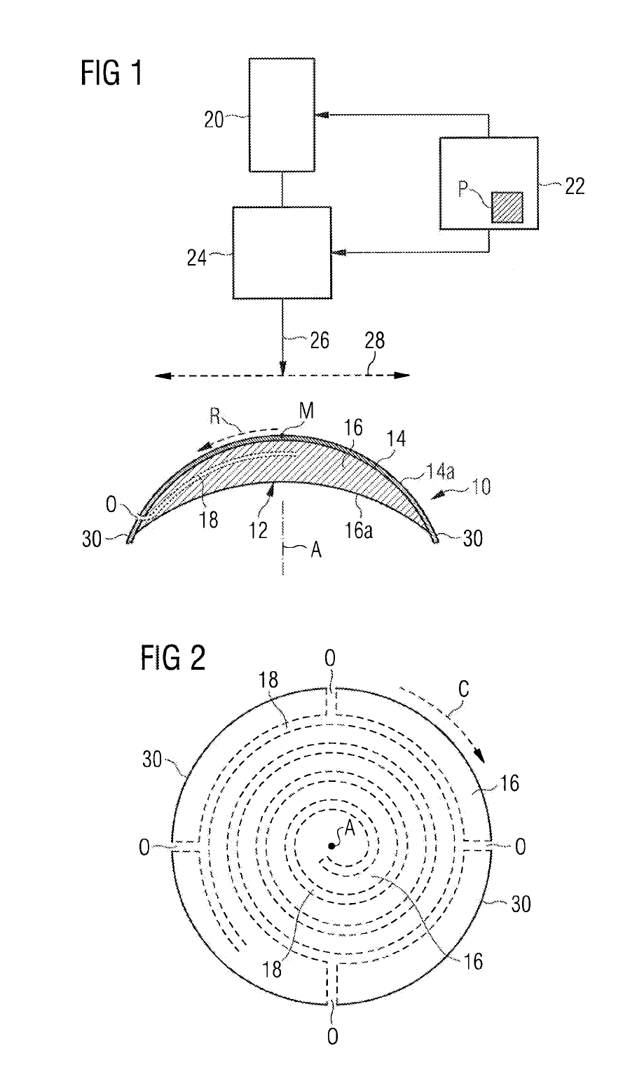

FIG. 1 shows schematically an eye 10 the biomechanical and/or biochemical proper ties of which are to be changed by introducing photosensitizer. This process is known as “corneal cross-linking.” For example, the mechanical stability of the cornea can be strengthened by the cross-linking. By using shaped bodies also the shape of the cornea can be changed during channel formation or cross-linking. In addition, the use of photosensitizers is also suitable for treating infectious inflammations of the cornea, the radicals which result killing off the germs there.

An eye axis is labeled “A” and this eye axis also very nearly coincides with the optical axis of the system for guiding and focusing laser radiation described in more detail below.

The center (midpoint) of the surface of the cornea (14a) is labeled “M”, so that a radial direction R can be defined starting from here. The eye tissue to be treated by cross-linking 12 is here essentially the cornea 16, which is covered externally by the epithelium 14.

Channels 18 are introduced into the stroma of the cornea 16 with the device to be described in more detail below. These channels 18 are in fluid-conducting contact with openings O (ports) and the openings O provide access from the outside into the channels for introducing photosensitizer solution. The channels 18 extend into the interior of the cornea 16 and terminate before its inner surface 16a.

A photosensitizer, e.g. riboflavin, is introduced into the channels 18 and penetrates into the channels and from there distributes itself in the corneal tissue by diffusion.

The device has a source 20 for laser radiation, e.g. a femtosecond laser, described above, such as is used e.g. for cutting a flap in femto-LASIK. As regards the system 24 for guiding and focusing the laser radiation 26 inside the cornea 16, systems which are already used in femto-LASIK are also appropriate here.

In contrast to LASIK a computer 22, which controls the laser radiation source 20 and the optical systems 24 for guiding and focusing the laser radiation 26, is programmed with a program P which controls the laser radiation 26 in a special way to create the channels 18. For this the laser radiation 26 undergoes a parallel displacement in the direction of the arrow 28 when creating the aforesaid channels 18 according to FIG. 1. The representation in FIG. 1 shows a view of the eye cut by a plane which contains the axis A. FIG. 1 also shows a channel 18 which extends substantially parallel to the surface 14a of the cornea. The channel is accessible from the outside via an opening O located near the limbus 30. A fine syringe can, for example, be introduced into the opening O so as to inject a photosensitizer solution or a gas, such as air, into the channel 18.

FIG. 2 shows a plan view of a cornea 16 and a channel 18 extending inside the cornea 16, this channel being in the form of a spiral in the embodiment depicted here and having additional openings O which are distributed at intervals in the peripheral direction C. The approximately spiral-shaped channels 18 are arranged in a plane which, in this embodiment, is substantially parallel to the surface 14a of the eye. As a variant of this embodiment the channels 18 may also be arranged in a plane which is perpendicular to the axis A. In a further variant the paths of the channels lie in a plane which is parallel to the rear surface 16a of the cornea 16. The choice of location and the path followed by the channels 18 can depend on the respective medical indication and can be chosen accordingly.

In the embodiment shown in FIG. 2 the channels are so positioned that they ensure that the photosensitizer distributes itself homogeneously by diffusion in the corneal tissue, at least in the space spanned by them.

As a modification of the embodiments shown in the figures, channels can also extend axially, i.e. parallel to the axis A, in part at least.

Channels can also extend radially.

Also, all the paths and arrangements of the channels described hitherto can be combined with one another at will.

Through the choice of the diameters and the geometric arrangement of the channels, the distribution of photosensitizer in the cornea can be controlled as desired, depending on the medical indication.

The channels are formed by focused laser radiation, in particular by means of a femtosecond laser, through cavitation bubbles created by the laser foci. In certain cases, adjacent cavitation bubbles do not overlap completely, so that some tissue remains between the individual cavitation bubbles. This tissue stabilizes the overall tissue in the structure while being sufficiently permeable as regards the diffusion of photosensitizer in the channels.

Instead of long channels it is also possible to create cavities with other shapes by means of the cavitation bubbles mentioned above, in particular planar cavities in which e.g., tissue regions spaced uniformly and dose together remain as “posts” between the upper and lower surfaces of the cavity or cavities.

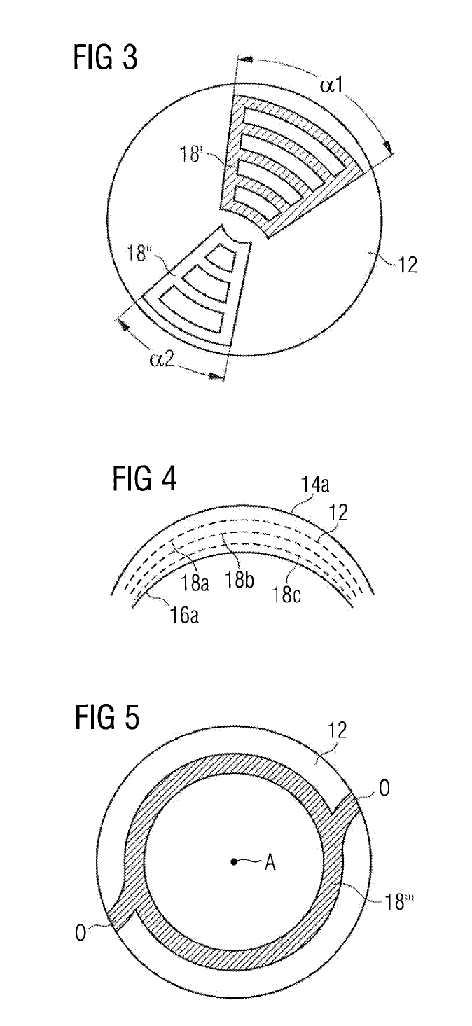

FIG. 3 shows an axial plan view of a cornea with a channel system 18′, 18″ with a contour which is shaped somewhat like the sector of a circle (as shown) for treating astigmatism. As is shown in FIG. 3, two sector-shaped channel systems 18′ and 18″ can be formed, each having a different sector angle α₁ and α₂.

FIG. 4 shows channels 18a, 18b, 18c which extend at different depths in relation to the surface 14a of the cornea. The three different depths for the channels shown schematically in FIG. 4 can be realized for all the structures and arrangements of channels described individually according to FIGS. 1, 2, 3 and 5 as well as other embodiments.

FIG. 5 shows a schematic plan view of a ring-shaped channel 18″ with two openings o which connect the channel 18″ with the surface of the cornea. As a variant of the embodiment according to FIG. 5, a plurality of ring-shaped channels can also be provided, which are connected either to one another and/or each individually with the surface of the cornea in a fluid-conducting state.

Certain embodiments also include a method for dissecting an eye for the introduction of photosensitizer where, by means of laser radiation 26 which is focused on and into the cornea, channels 18 are created in the cornea which extend from the surface 14a of the cornea into the interior of the cornea. In this method all the characteristics and properties of the channels 18 which have been described above can be employed.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

1. A device for the introduction of photosensitizer into eye tissue, the device comprising:
a source for laser radiation;
a system for guiding and focusing the laser radiation with respect to the eye tissue; and
a computer for controlling the system, the computer being programmed to control the laser radiation to create in the eye tissue at least one or more channels that extends at least partially into the interior of the eye tissue wherein the at least one or more channels are connected to one or more openings in the surface of the eye tissue.

2. The device of claim 1 wherein the at least one or more channels or the plurality of channels are oriented essentially transversely to the axis of the eye.

3. The device of claim 1 wherein the computer is programmed to create one or more channels into the eye tissue along paths that are oriented essentially radially.

4. The device of claim 1 wherein the one or more channels penetrate essentially the radial area of the cornea with essentially uniform channel density.

5. The device of claim 1 wherein at least one of the one or more channels follows a spiral path.

6. The device of claim 1 wherein the one or more channels are at least partially created by the cavitation bubbles created by the laser radiation and at least in part do not merge completely with one another, the distance between adjacent cavitation bubbles being in the range from 1 to 50 pm.

7. The device of claim 1 wherein at least one of the one or more channels follows a path that is at least partly axial and/or at least partially curved.

8. The device of claim 1 wherein the computer is programmed to create in the eye tissue a plurality of channels whose density varies according to the position in the eye tissue.

9. The device of claim 1 wherein the computer is programmed to create in the eye tissue a plurality of channels whose depth and/or cross-section varies according to the position in the eye tissue.

10. The device of claim 1 wherein the computer is programmed to create in the eye tissue a plurality of channels wherein at least two different channels have different cross-sections.

11. The device of claim 1 wherein the source for laser radiation is a femtosecond laser, a nanosecond laser, an attosecond laser, or a picosecond laser.

12. The device of claim 1 wherein the computer is programmed to create in the eye tissue a plurality of channels with a shape that corresponds approximately to that of the sectors of a circle for treating astigmatism.

13. The device of claim 1 wherein the computer is programmed to create one or more channels that follow a path that is at least approximately ring-shaped.

14. The device of claim 1 wherein the computer is programmed to create in the eye tissue a plurality of channels that extend to different depths in the eye tissue.

15. A method for introducing a photosensitizer into a cornea of an eye, the method comprising:
providing laser radiation;
guiding and focusing the laser radiation with respect to the cornea; and
controlling the laser radiation such that it creates in the cornea a plurality of channels which extend in the interior of the cornea wherein the laser radiation is controlled such that the plurality of channels are connected to openings in the surface of the cornea.

16. A method for performing refractive surgery at a cornea of a patient, the method comprising the steps of:
providing a first laser radiation;
guiding and focusing said first laser radiation with respect to the cornea and controlling the first laser radiation such that it creates in the cornea channels which extend in the interior of the cornea;
introducing a sensitizer into said channels for hardening corneal tissue;
providing a second laser radiation; and
guiding the second laser radiation onto an exposed surface of the cornea for ablation of corneal tissue.

17. A method for performing refractive surgery at a cornea of a patient, the method comprising the steps of:
introducing a sensitizer into the cornea;
hardening the cornea by means of the sensitizer; and
guiding laser radiation onto said cornea for ablation of corneal tissue.

18. A system for corneal surgery comprising:
one or more sources generating first and second laser radiation;
optical elements guiding and focusing said first and second laser radiation;
a computer programmed to control said optical elements relative to corneal tissue of an eye wherein said first laser radiation generates in said corneal tissue one or more channels that extend at least partially into an interior of the cornea and wherein said second laser radiation impinges onto an exposed surface of the cornea for ablation of corneal tissue or said second laser radiation cuts a flap in the cornea.
